(19) United States
(12) Patent Application Publication
(10) Pub. No.: US 2020/0399204 A1
(43) Pub. Date: Dec. 24, 2020

(54) PHOTO ACTIVATOR AND MANUFACTURING METHOD OF THE SAME AND PHOTO ALIGNMENT AGENT INCLUDING THE SAME

(71) Applicants: Dong Myung Shin, Seoul (KR); HONGIK UNIVERSITY INDUSTRY-ACADEMIC COOPERATION FOUNDATION, Seoul (KR)

(72) Inventors: Dong Myung SHIN, Seoul (KR); In Hye LEE, Seoul (KR)

(21) Appl. No.: 16/962,574

(22) PCT Filed: Jan. 18, 2018
(86) PCT No.: PCT/KR2018/000820

§ 371 Date: Jul. 16, 2020

(51) Int. Cl. C07C 249/12; C09K 19/56

(52) U.S. Cl. CPC C07C 249/12; C07C 251/44; C09K 19/56

(57) ABSTRACT

The present disclosure relates to the photoactive agent having an N—O—C═O—C band and being mixed with a liquid crystal aligning agent, a manufacturing method of photoactive agent comprising a primary solution preparation step for preparing a primary solution by mixing and dissolving cyclohexanone oxime in a solvent, a secondary solution manufacturing step for preparing a secondary solution by mixing anhydride in the primary solution, a reaction step for proceeding a synthesis reaction of the photoactive agent in a reaction solution in which a catalyst is mixed with the secondary solution, and an extraction step for extracting the photoactive agent in the reaction process.

### TECHNICAL FIELD

The present invention relates to a photoactive agent layer for improving liquid crystal alignment, a manufacturing method of the same, and a manufacturing method of a liquid crystal alignment agent comprising the same.

### BACKGROUND ART

The liquid crystal cell of the liquid crystal display device is configured by filling the liquid crystal between the substrates facing each other. In the liquid crystal cell, a liquid crystal alignment layer is coated on a substrate surface in order to align liquid crystal molecules in a predetermined direction with respect to the substrate surface.

However, since the rubbing process tends to generate dust or static electricity during the rubbing process, dust may adhere to the surface of the liquid crystal alignment layer and cause display defects. In addition, the circuit of the TFT device may be destroyed by static electricity generated by friction in the rubbing process, which may cause a decrease in product yield. In addition, in the liquid crystal cell, problems such as image sticking due to low anchoring energy of the liquid crystal alignment layer may cause deterioration of image quality.

Recently, a method of forming a liquid crystal alignment layer in multiple layers or a method of implanting ions into the liquid crystal alignment layer has been attempted to solve problem such as image sticking. However, this method has a problem that productivity and efficiency are reduced due to a long process time.

On the other hand, the photo-alignment method can solve many problems in the process caused by rubbing, and has an advantage in widening a viewing angle in a large liquid crystal display. However, the photo-alignment method is that the light irradiation time is long, the amount of light is too large, the energy consumption is large, and the fixed energy is low, resulting in image sticking. In addition, recently, since the inside of the process chamber is contaminated by organic by-products generated by the photo-alignment method, there is a problem in that the process chamber must be periodically cleaned.

### DISCLOSURE OF THE INVENTION

### Technical Problem

The present invention provides a photoactive agent capable of efficiently forming a liquid crystal alignment film, a manufacturing method of the same, and a manufacturing method of a liquid crystal alignment agent comprising the same.

### Technical Solution

The photoactive agent of the present invention is characterized by having an N—O—C═O—C band, and being mixed with a liquid crystal alignment agent.

Further, the photoactive agent layer may have a structural formula according to FIGS. 1 and 2.

In addition, the photoactive agent may be formed by the reaction of cyclohexanoneoxime and crotonic anhydride, or may be formed by the reaction of cyclohexanoneoxime and acetic anhydride.

The method for preparing a photoactive agent of the present invention is characterized in that it comprises a primary solution preparation process for mixing and dissolving cyclohexanone oxime in a solvent to prepare a primary solution, and a secondary solution preparation process for mixing anhydride in the primary solution to prepare a secondary solution, and a reaction process for performing a synthesis reaction of a photoactive agent in a reaction solution in which a catalyst is mixed with the secondary solution, and an extraction process for extracting the photoactive agent in the reaction process.

In addition, the solvent may include any one selected from the group consisting of hexane THF, DMF and DMSO.

In addition, the anhydride may be crotonic anhydride when the solvent is hexane, and acetic anhydride when the solvent is dimethyl formamide.

In addition, the catalyst may be perchloric acid, sulfuric acid, para-toluenesulfonic acid, hydrochloric acid or nitric acid.

In addition, in the extraction process, when the solvent is hexane, the photoactive agents can be extracted after the reaction solution in which the synthesis reaction is completed is washed several times with hexane and deionized water by being alternately mixed with hexane and deionized water, and separated into a layer of hexane and a layer of deionized water. Further, in the extraction process, when the solvent is dimethyl formamide, the photoactive agents can be extracted after the reaction solution is separated into an ethyl acetate layer and a layer of deionized water by mixing with ethyl acetate in the reaction solution and diluted the reaction solution to remove the dimethyl formamide, and by repeatedly mixing with deionized water in the reaction solution to wash the ethyl acetate layer and the ethyl acetate is evaporated.

In addition, the extraction process is performed in a state in which light is blocked, and the extracted photoactive agent may be stored in a vacuum by sealing it in a container in which light and oxygen are not transmitted.

The manufacturing method of a liquid crystal alignment agent of the present invention comprises a photoactive agent mixing step of mixing the photoactive agent in the alignment undiluted solution in a predetermined ratio, and a pyrrolidinone mixing step of mixing pyrrolidinone in the photoactive agent mixed solution, and 2-butoxyethanol mixing step of mixing 2-butoxyethanol in the photoactive agent mixture solution, and gamma-butyrolactone mixing step of mixing gamma-butyrolactone in the photoactive agent mixture solution and a stirring step of preparing the liquid crystal alignment agent by stirring the photoactive agent mixed solution for a predetermined time after the gamma-butyrolactone mixing step

In addition, the alignment undiluted solution may be TN-PAA or P-PAA.

In addition, the alignment undiluted solution may be a photodegradable polymer resin.

### Advantageous Effects

The present invention has an effect of forming a liquid crystal alignment layer very quickly and efficiently by shortening the light irradiation time within a few minutes, unlike the need for a light irradiation time and a very large amount of light, which take more than 10 minutes since the present invention forms a liquid crystal alignment layer through coating and ultraviolet irradiation after making a liquid crystal aligning agent by mixing a photoactive agent sensitive to ultraviolet rays.

In addition, the present invention has an effect that the post-process is simplified, and the process chamber can be easily managed compared to the existing process since the present invention does not generate other volatile substances except carbon dioxide in the process of forming a liquid crystal alignment layer.

In addition, the present invention is photo-aligned by radicals formed by the photoactive agent in the light irradiation process and has the effect that the surface area is increased and the surface energy is increased to increase the fixed energy of the liquid crystal.

### BRIEF DESCRIPTION OF THE DRAWINGS

FIG. 1 is a reaction formula of the photoactive agent of the structural formulas (a) and (b) to generate radicals by ultraviolet light.

FIG. 2 is a process chart for a manufacturing method of a photoactive agent according to an embodiment of the present invention.

FIG. 3 is a reaction scheme according to a manufacturing method for preparing a photoactive agent according to an embodiment of the present invention.

FIG. 4 is a graph of H-NMR measurement of a photoactive agent according to an embodiment of the present invention.

FIG. 5 is an IR spectrum measurement graph of a photoactive agent AAP according to an embodiment of the present invention.

FIG. 6 is an IR spectrum measurement graph of a photoactive agent CAP according to an embodiment of the present invention.

FIG. 7 is a UV-Visible Spectrum measurement graph according to the ultraviolet irradiation time of the photoactive agent AAP according to an embodiment of the present invention.

FIG. 8 is a UV-Visible Spectrum measurement graph according to the ultraviolet irradiation time of the photoactive agent AAP according to an embodiment of the present invention.

FIG. 9 is a process chart for a manufacturing method of manufacturing a photo alignment agent according to an embodiment of the present invention.

FIG. 10 is a configuration diagram of an optical system for measuring a fixed energy of a liquid crystal alignment layer.

FIG. 11 is a configuration diagram of an optical system for measuring a pretilt angle of a liquid crystal alignment layer.

FIG. 12 is a polar diagram of a liquid crystal alignment layer by a photo alignment agent according to an embodiment of the present invention.

FIG. 13 is an AFM photograph of a surface before and after light irradiation of a liquid crystal alignment layer by a photo alignment agent according to an embodiment of the present invention.

### BEST MODE FOR CARRYING OUT THE INVENTION

Hereinafter, a photoactive agent, a manufacturing method for the same, and a manufacturing method of a liquid crystal alignment agent including the same according to an embodiment of the present invention, is described in detail with reference to the accompanying drawings and examples.

First, a photoactive agent according to an embodiment of the present invention will be described.

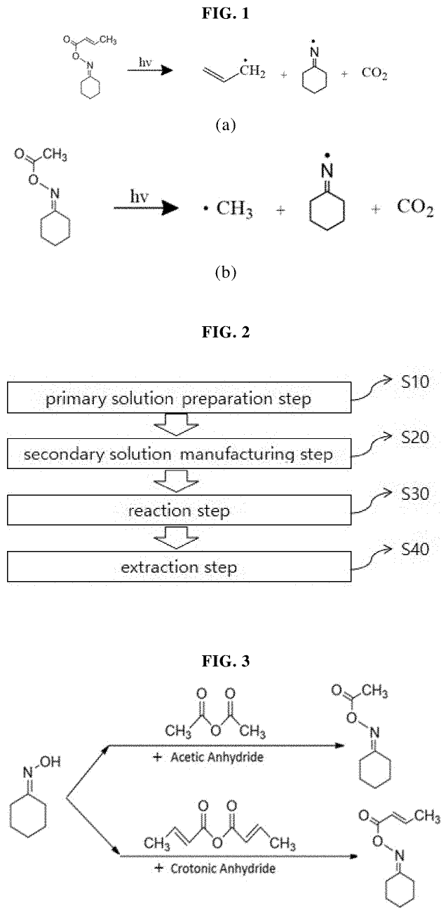

FIG. 1 is a reaction formula of the photoactive agent of the structural formulas (a) and (b) to generate radicals by ultraviolet light.

The photoactive agent may be formed of a material represented by the following structural formula (a). The photoactive agent may be a photoactive agent (hereinafter referred to as “CAP”) formed by the reaction of cyclohexanoneoxime and crotonic anhydride.

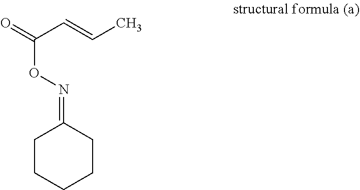

In addition, the photoactive agent may be formed of a material represented by the following structural formula (b). The photoactive agent may be a photoactive agent (hereinafter referred to as “AAP”) formed by the reaction of cyclohexanoneoxime and acetic anhydride.

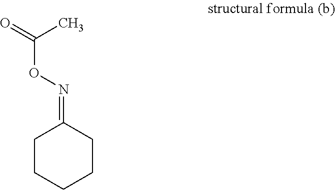

As shown in structural formulas (a) and (b), the photoactive agent has an N—O—C═O—C band in common. The photoactive agent has great reactivity under the influence of the N—O—C═O—C band. More specifically, as shown in FIG. 1, when light (ultraviolet rays) is irradiated, the photoactive agent decomposes into a plurality of radicals within 30 seconds to 10 minutes, and thus has a relatively large reactivity. The photoactive agent releases only carbon dioxide in the decomposition process.

In addition, the photoactive agent may be formed of a material represented by the following structural formula (c), (d) or (f). The photoactive agent has a N—O—C═O—C band and decomposes into a plurality of radicals when light (ultraviolet rays) is irradiated, and a new bond between the radical and polyamic acid (PAA) has liquid crystal directionality which is dependent on the size of the alkyl group having the radical. In addition, the photoactive agent may be made to have a liquid crystal alignment within a short time by generating a bond while reacting with polyimide.

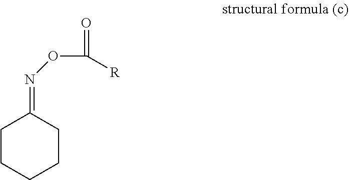

(Wherein R is alkyl group or cyclohexyl group of CₙH₂ₙ₊₁ having 1 to 12 carbon atoms, and n is a positive integer.)

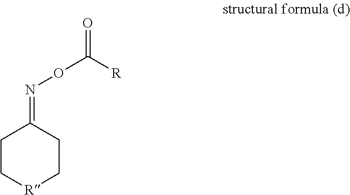

(Wherein R is a structure represented by the following structural formula (e) or a cyclohexyl group, R″ is alkyl group of CₙH₂ₙ₊₁ having 1 to 5 carbon atoms, and n is a positive integer.)

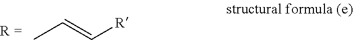

(Wherein R′ is alkyl group of CₙH₂ₙ₊₁ having 1 to 12 carbon atoms, n is a positive integer, and R and R″ are the same or different from each other.)

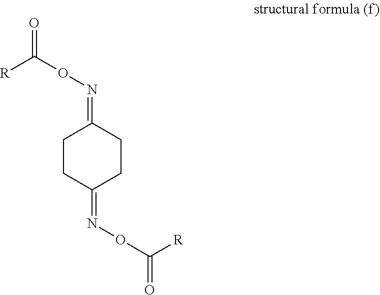

(Wherein R is alkyl group or a cyclohexyl group of CₙH₂ₙ₊₁ having 1 to 12 carbon atoms, and n is a positive integer.)

The photoactive agent is mixed with polyamic acid (PAA) in a certain ratio and forms a liquid crystal alignment agent. The liquid crystal alignment agent forms a liquid crystal alignment layer, and is improved liquid crystal alignment property by light irradiation. That is, the photoactive agent may improve liquid crystal alignment property of the liquid crystal alignment layer. The polyamic acid may be a TN (Twist-nematic) horizontal alignment agent or TN horizontal alignment polyamic acid (hereinafter referred to as “TN-PAA”) conventionally used for forming a liquid crystal alignment layer) and a photodegradable alignment agent or photodegradable polyamic acid (hereinafter referred to as “P-PAA”). On the other hand, the TN-PAA forms a TN-Polyimide layer (hereinafter referred to as “TN-PI”) which is conventional liquid crystal alignment layer, and the P-PAA forms a P-Polyimide layer (hereinafter referred to as “P-PI”) which is conventional liquid crystal alignment layer through a reaction in state the photoactive agent is not mixed.

Next, a manufacturing method of a photoactive agent according to an embodiment of the present invention will be described.

FIG. 2 is a process chart for a manufacturing method of a photoactive agent according to an embodiment of the present invention. FIG. 3 is a reaction scheme according to a method for preparing a photoactive agent according to an embodiment of the present invention

Referring to FIG. 2 and FIG. 3, the method for manufacturing a photoactive agent according to an embodiment of the present invention includes a first solution manufacturing step S10, a second solution manufacturing step S20, a reaction step S30, and an extraction step S40. Hereinafter, a description will be given based on a method of manufacturing a CAP, and another part of the manufacturing method will be further described in a manufacturing method of an AAP.

The primary solution manufacturing step S10 is a process of preparing a primary solution by mixing and dissolving cyclohexanone oxime in a solvent. The solvent may be hexane. In addition, the solvent may be a polar aprotic solvent. For example, the solvent may be tetrahydrofuran (THF), dimethylformamide (DMF), or dimethylsulfoxide (DMSO). Meanwhile, when preparing the AAP, dimethylformamide may be used as a solvent. The primary solution is prepared by mixing cyclohexanone oxime in an amount of a solvent capable of sufficiently dissolving cyclohexanone oxime. For example, the primary solution may be prepared by dissolving 0.01 mole of cyclohexanone oxime in 50 ml of hexane as a solvent. If the amount of the solvent is too small, cyclohexanone oxime may not be sufficiently dissolved. If the amount of the solvent is too large, the time for evaporating the solvent in the subsequent process may be increased.

The primary solution manufacturing step S10 may be performed while mixing a solvent and cyclohexanone oxime in a container such as a flask, followed by stirring using a stirrer. At this time, the stirrer can be stirred for 10 to 30 minutes at 40 to 60 rpm.

The secondary solution manufacturing step S20 is a process of preparing a secondary solution by mixing anhydride with the primary solution. As the anhydride, crotonic anhydride may be used. Meanwhile, in the case of preparing the AAP, acetic anhydride may be used as the anhydride. The anhydride may be mixed into the primary solution at 0.010 to 0.012 moles. If the content of the anhydride is too small, the anhydride may remain unreacted without reacting with cyclohexanone oxime. In addition, if the content of the anhydride is too large, cyclohexanone oxime may remain unreacted. The secondary solution manufacturing step S20 may be performed while stirring using a stirrer. At this time, the stirrer can be stirred for 10 to 30 minutes at 40 to 60 rpm.

The reaction step S30 is a process of proceeding a synthesis reaction of the photoactive agent in a reaction solution in which the catalyst is mixed with the secondary solution. Perchloric acid may be used as the catalyst. In addition, an acid such as sulfuric acid, p-toluenesulfonic acid, hydrochloric acid or nitric acid may be used as the catalyst. The catalyst may be mixed in 2-3 drops when the solvent is 50 ml. The reaction step S30 may be performed while stirring the reaction solution using a stirrer. At this time, the stirrer can be maintained at 40˜60 rpm. The reaction step S30 may be carried out at 20˜25° C. The reaction process can be carried out for 20 to 30 hours. In addition, the reaction process S30 may be performed in a state in which the reaction solution is not exposed to light.

The extraction step S40 is a process of extracting a photoactive agent from the reaction solution after the synthesis reaction is completed. First, hexane and deionized water are alternately mixed with the reaction solution in which the synthesis reaction is completed, the reaction solution is washed several times with hexane and deionized water, and the reaction solution is separated into a hexane layer and a deionized water layer. Deionized water remaining in the separated hexane layer may be partially removed using sodium sulfate or anhydrous calcium chloride. Additionally, hexane can be evaporated from the water and agglomerated mass. For example, after filtering the water and agglomerated mass first using a paper filter and a funnel, hexane is evaporated using an evaporator to extract the photoactive agent.

On the other hand, in the case of preparing the AAP, first, 10 times or more ethyl acetate is mixed and diluted in the reaction solution to remove dimethyl formamide. At this time, the reaction solution is maintained for a sufficient time and repeated several times to remove all of dimethyl formamide. If the amount of the solvent is too large, the time for diluting the solvent in the subsequent process may increase. Next, the deionized water was repeatedly mixed and washed in the reaction solution, and the reaction solution is separated into the ethyl acetate layer and the deionized water layer. The photoactive agent is extracted from ethyl acetate layer by evaporating ethyl acetate using an evaporator.

The extracted photoactive agent is a light brown liquid with a low viscosity. Since the photoactive agent has changed properties when it comes into contact with oxygen or light, it is sealed and stored in a container that does not transmit oxygen and light. For example, the photoactive agent can be stored in a vacuum by sealing it with a fly layer in a brown vial bottle.

Next, evaluation of the properties of the photoactive agent according to an embodiment of the present invention will be described.

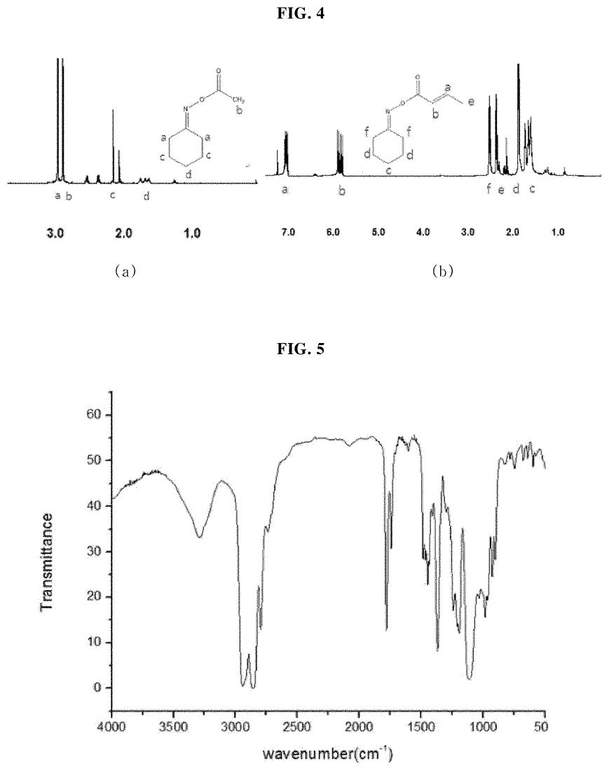

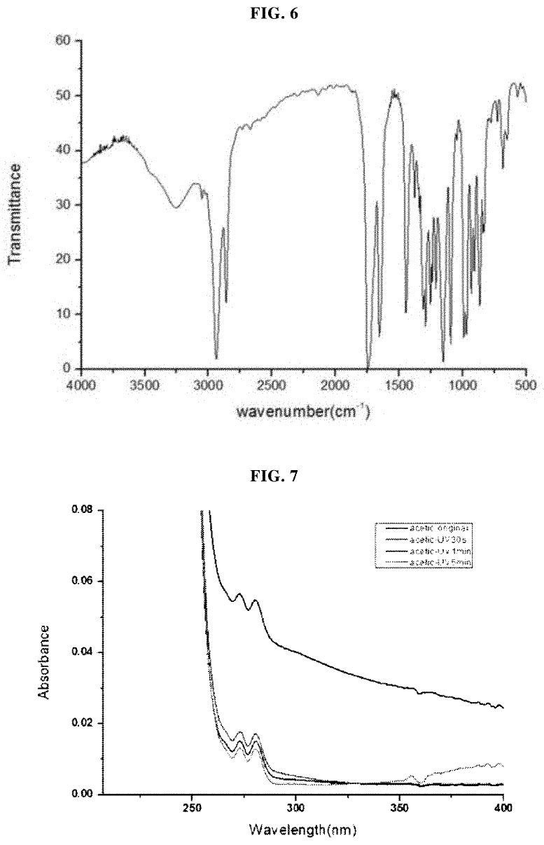

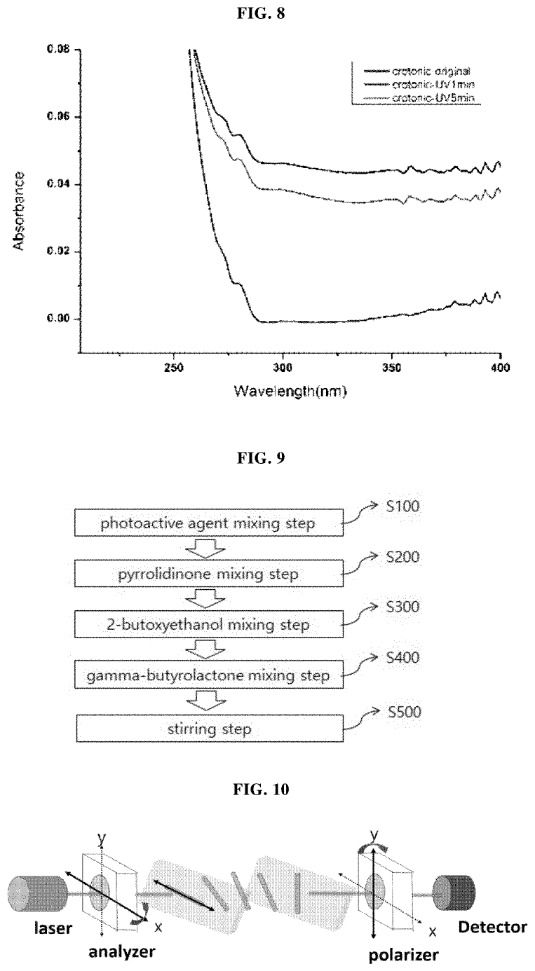

FIG. 4 is a graph of H-NMR measurement of a photoactive agent according to an embodiment of the present invention. FIG. 5 and FIG. 6 are an IR spectrum measurement graph of a photoactive agent according to an embodiment of the present invention. FIG. 7 and FIG. 8 are a UV-Visible Spectrum measurement graph according to the ultraviolet irradiation time of the photoactive agent according to an embodiment of the present invention.

The photoactive agent was evaluated through H-NMR measurement, IR spectrum measurement, and UV-Visible spectrum measurement according to ultraviolet irradiation time.

FIG. 4(a) is a graph of H-NMR measurement of AAP, and FIG. 4(b) is a graph of H-NMR measurement of CAP.

Referring to FIGS. 4(a) and 4(b), According to the H-NMR measurement result, the photoactive agent may be confirmed to have a peak corresponding to the photoactive agent of the structural formula (a) and (b). The photoactive agent has a specific peak at 270 to 280 nm for both AAP and CAP.

FIG. 5 is an IR spectrum measurement graph of the AAP, and FIG. 6 is an IR spectrum measurement graph of the CAP.

Referring to FIGS. 5 and 6, According to the IR Spectrum measurement results, it can be seen that the photoactive agent has an IR peak corresponding to the structural formula of the photoactive agent of FIG. 1.

FIG. 7 is a UV-Visible Spectrum measurement graph according to the UV irradiation time of the AAP, FIG. 8 is a UV-Visible Spectrum measurement graph according to the UV irradiation time of the CAP.

Referring to FIGS. 7 and 8, according to the UV-Visible Spectrum measurement result according to the ultraviolet irradiation time, the photoactive agent decreases the peaks observed in the graph of FIG. 4 as the UV irradiation time increases. Therefore, it can be confirmed that the photoactive agent is highly reactive.

Next, a method of manufacturing a liquid crystal aligning agent according to an embodiment of the present invention will be described.

FIG. 9 is a process chart for a manufacturing method of manufacturing a photo alignment agent according to an embodiment of the present invention.

Referring to FIG. 9, the liquid crystal alignment agent manufacturing method may include a photoactive agent mixing step S100, a pyrrolidinone mixing step S200, a 2-butoxyethanol mixing step S300, a gamma-butyrolactone (γ-butyrolactone) mixing step S400 and a stirring step S500. The liquid crystal alignment agent manufacturing method is a manufacturing method of a liquid crystal alignment agent using the photoactive agent described above.

The photoactive agent mixing step S100 is a process of preparing a photoactive agent mixed solution by mixing a photoactive agent in an alignment undiluted solution with a predetermined ratio. Various materials used to form the liquid crystal alignment layer may be used as. Polyimide resin or polyvinyl alcohol (PVA) may be used as the alignment undiluted solution. As the polyimide resin, polyamic acid or soluble polyimide may be used. The polyamic acid may be TN-PAA or photodegradable polyamic acid (P-PAA). The alignment undiluted solution may preferably be polyamic acid.

The photoactive agent according to an embodiment of the present invention is used as the photoactive agent. The alignment undiluted solution and the photoactive agent may be mixed by the mixing ratio of 1:0.1 to 1:3 in a weight ratio. The mixing ratio of the alignment undiluted solution and the photoactive agent may be varied within the range of the mixing ratio according to the type of the alignment undiluted solution and the photoactive agent. For example, when the alignment undiluted solution is TN-PAA, the photoactive agent mixed solution may be formed by mixing 0.1 g of the photoactive agent with 1.0 g of polyamic acid. In addition, the photoactive agent mixed solution may be formed by mixing 1.5 g of a photoactive agent with 1.0 g of polyamic acid. The polyamic acid and the photoactive agent are supplied to a light-blocking mixing container, for example, a brown vial and mixed with each other. The photoactive agent mixing step is carried out by stirring the mixed solution at a rate of 20 to 40 rpm at a temperature of 4 to 10° C. to prevent oxidation and decomposition of the polyamic acid. The photoactive agent mixing step may be performed for approximately 1 to 5 minutes.

Meanwhile, the photoactive agent mixing step S100 may be mixed after mixing other raw materials. During the mixing step of the photoactive agent, when it is difficult to block the photoactive agent from light and oxygen, or when the photoactive agent cannot be reduced in contact with light and oxygen, the photoactive agent may be mixed in the last step. For example, the photoactive agent mixing step S100 may be performed after the gamma-butyrolactone mixing step S400.

The pyrrolidinone mixing step S200 is a process of mixing pyrrolidinone with a photoactive agent mixed solution. The pyrrolidinone may be N-methyl-2-pyrrolidinone (NMP). Since the pyrrolidinone has excellent solubility in polyamic acid, the pyrrolidinone allows the polyamic acid and the photoactive agent to be mixed well. The pyrrolidinone may be mixed in a mixing ratio of 1:0.1 to 1:0.4 based on the weight of the alignment undiluted solution. For example, the pyrrolidinone may be formed by mixing 0.2 g with respect to 1.0 g of polyamic acid. Alternatively, the photoactive agent mixed solution and pyrrolidinone may be mixed in a ratio of 3:1 to 8:1 by weight ratio. The pyrrolidinone mixing step is performed by rotating the mixing vessel at a speed of about 20 to 40 rpm at room temperature. The pyrrolidinone mixing step may be performed for approximately 1 to 5 minutes.

The 2-butoxyethanol mixing step S300 is a process of mixing 2-butoxyethanol in a photoactive agent mixture solution. The 2-butoxyethanol reduces the surface tension of the liquid crystal alignment agent so that the thin film is uniformly formed during spin coating of the liquid crystal alignment agent. The 2-butoxyethanol may be mixed in a ratio of 0.1 to 1:0.4 based on the weight of the alignment undiluted solution. For example, the 2-butoxyethanol may be formed by mixing 0.2 g with respect to 1.0 g of polyamic acid. Alternatively, the photoactive agent mixture solution and 2-butoxyethanol may be mixed at a weight ratio of 9:1 to 25:1. The 2-butoxyethanol mixing step S300 is performed by stirring the mixed solution at a rate of about 20 to 40 rpm at room temperature. The 2-butoxyethanol mixing step may be performed for approximately 1 to 5 minutes.

The gamma-butyrolactone mixing step S300 is a process of mixing gamma-butyrolactone in the photoactive agent mixed solution. The gamma-butyrolactone is excellent in solubility in the polyamic acid so that the polyamic acid and the photoactive agent are well mixed. The gamma-butyrolactone may be mixed in a ratio of 0.3 to 1:0.7 based on the weight of the alignment undiluted solution. For example, the gamma-butyrolactone may be mixed by 0.4 g with respect to 1.0 g of the polyamic acid. The photoactive agent mixed solution and gamma-pyrrolidinone may be mixed in a ratio of 3:1 to 6:1 by weight ratio. The gamma-butyrolactone mixing step is carried out by rotating the mixing container at room temperature at a rate of 20 to 40 rpm. The gamma-butyrolactone mixing step may be performed for approximately 1 to 5 minutes.

The stirring step S500 is a process of preparing a liquid crystal alignment agent by stirring the photoactive agent mixed solution for a predetermined time after the gamma-butyrolactone mixing step S300. The stirring step may be performed at a temperature of 4˜10° C. at a speed of 20˜40 rpm for a minimum of 6 hours.

The liquid crystal alignment agent according to the method for manufacturing the liquid crystal alignment agent may be coated on a substrate with a predetermined thickness to form a liquid crystal alignment layer. For example, the liquid crystal alignment layer may be formed to a thickness of 50 to 200 nm.

Next, the results of evaluating the properties of the liquid crystal alignment layer produced by the manufacturing method of a liquid crystal alignment agent according to an embodiment of the present invention will be described.

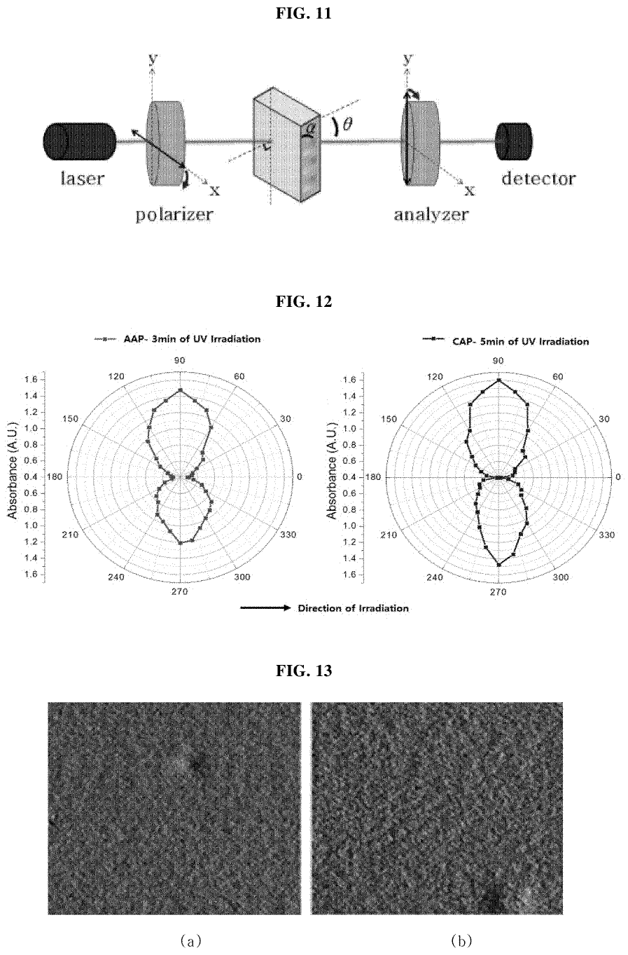

FIG. 10 is a configuration diagram of an optical system for measuring a fixed energy of a liquid crystal alignment layer. FIG. 11 is a configuration diagram of an optical system for measuring a pretilt angle of a liquid crystal alignment layer. FIG. 12 is a polar diagram of a liquid crystal alignment layer by a photo alignment agent according to an embodiment of the present invention. FIG. 13 is an AFM photograph of a surface before and after light irradiation of a liquid crystal alignment layer by a photo alignment agent according to an embodiment of the present invention.

An ITO glass substrate was used as a substrate. The glass substrate was ultrasonically cleaned using deionized water in which mucarsol was mixed in advance. In addition, the glass substrate was washed with acetone, IPA, and deionized water, followed by nitrogen cleaning.

The liquid crystal alignment layer was formed by spin coating a liquid crystal aligning agent on the glass substrate. The spin coating was performed in two steps by proceeding at a speed of 500 to 1,000 rpm for 10 seconds and at a speed of 1,500 to 3,000 rpm for 20 seconds for a predetermined thickness. TN-PAA and P-PAA of polyamic acid were used as the alignment undiluted solution. The liquid crystal alignment layer was baked at a predetermined temperature for a predetermined time. For example, the liquid crystal alignment layer was baked at 80° C. for 2 minutes and at 200° C. for 15 minutes when TN-PAA was used as the liquid crystal alignment layer, and at 90° C. for 10 minutes and at 230° C. for 30 minutes when P-PAA was used as the liquid crystal alignment layer. For the photo-alignment of the liquid crystal alignment layer, ultraviolet light was irradiated using a 1000 W intensity ultraviolet source and a 310 to 400 nm polarizer. The intensity of ultraviolet light passing through the polarizer was measured to be 1.5 mW/cm2 (UV-340, Lutron). The irradiation time of ultraviolet light was set at intervals of 2 minutes for 1 to 10 minutes. The two glass substrates required to make the liquid crystal cell were all manufactured under the same conditions, and were bonded in the anti-parallel direction. A 5.5 μm spacer and a UV curing agent were used to form a gap in the liquid crystal cell. The smaller the gap of the liquid crystal cell, the larger the error value of the anchoring energy, so that the cap of the liquid crystal cell was not made small.

In the embodiment in which both the rubbing process and the photo-alignment process were performed in the evaluation process, the rubbing process was performed before the photo-alignment process, and each glass substrate maintained a vacuum state before the rubbing process. The rotating speed of the rubbing cloth was set to 1200 rpm, and the moving speed of the glass substrate was set to 500 rpm. When the photo-alignment process was performed after the rubbing process, the polarizing plate was placed perpendicular to the rubbing direction and irradiated with ultraviolet light to make the light alignment in the same direction as rubbing. The liquid crystal alignment layer for this evaluation was prepared by varying the photoactive agent, polyamic acid type, and light irradiation time.

The property evaluation items of the liquid crystal alignment layer were fixed energy (anchoring energy), pretilt angle, dichroic ratio and polar-diagram. In addition, the surface roughness of the liquid crystal alignment layer was also evaluated.

The fixed energy is measured using a polarizer and an analyzer attached to rotation stages 1 and 2 spaced apart from each other, as the optical system shown in FIG. 10, and a LABview program that operates them. The liquid crystal cell was located between the polarizing plate and the analyzing plate. The polarizing plate and the analytical plate were rotated by 0.1 degrees to 1 degree in opposite directions to each other, and angles representing minimum transmittance (minimum mV) were measured. In the measurement of the fixed energy, a mixture of MLC-0643 liquid crystal mixed with 0.33 wt % chiral dopant (R-811, Merck) was injected into the liquid crystal cell. The fixed energy was measured using the difference in angle due to the force caused by the dopant and the force caused by the photo alignment. The initial mV when the optical system was installed was approximately 0.6 mV in the dark state, and the dark state was maintained as much as possible during the measurement process.

The pretilt angle was measured using a polarizer rotation method, and the optical system for measurement was configured as shown in FIG. 11. The optical system for the pretilt angle was installed in the same manner as in the case of fixed energy measurement, and the liquid crystal cell was fixed in a 50-degree twisted state. In the measurement of the pretilt angle, only MLC-0643 (Δε=6.9, Δn=0.1023, Merck) liquid crystal was injected into the liquid crystal cell. The pretilt angle was calculated using the Extended Jones Matrix after measuring the angle at the minimum transmittance while rotating the polarizer and the analyzer in opposite directions as in the case of fixed energy. At this time, the no value for calculating the pretilt angle was 1.4856 in the case of MLC-0643.

The dichroic ratio and polarity were measured using UV-Vis while rotating the polarizing plate by 10 degrees. Disperse Blue 14 was used to measure the dichroic ratio and polarity, and absorbance at 655 nm was measured. The dichroic ratio was calculated using the Maier-Saupe theory and by the DR=(A_{perpenclicular}−Aₚₐᵣₐₗₗₑₗ/A_{perpendicular}+Aₚₐᵣₐₗₗₑₗ) formula. The dichroic ratio and polarity indicate the degree of alignment of the liquid crystal alignment layer.

The surface roughness of the liquid crystal alignment layer was evaluated using an AFM (Atomic Force Microscope). The surface roughness of the liquid crystal alignment layer was evaluated in comparison with before and after light irradiation.

On the other hand, in order to relatively compare the results of evaluation of the properties of the liquid crystal alignment layer, the same evaluation was performed for the conventional liquid crystal alignment layer. That is, after performing only the rubbing process on the conventional liquid crystal alignment layers that is TN polyimide and Photo polyimide, the fixed energy and the pretilt angle were measured. The conventional liquid crystal alignment layer may be used as a reference for comparing the results of the liquid crystal alignment layer according to the present invention.

Table 1 shows measurement results of fixed energy for the conventional liquid crystal alignment layer. In the liquid crystal alignment layer, which has been previously subjected to only the rubbing process, fixed energy is measured to be 1.110×10⁻⁵ J/m² and 1.655×10⁻⁵ J/m², respectively.

TABLE 1

| liquid crystal alignment layer | fixed energy (×10⁻⁵ J/m²) |
|---|---|
| TN-polyimide(TN-PI) | 1.110 |
| Photo-polyimide(P-PI) | 1.655 |

Table 2 shows fixed energy evaluation results for the liquid crystal alignment layer according to the embodiment of the present invention. The fixed energy (×10⁻⁵ J/m²) was evaluated by varying the photoactive agent, polyamic acid and light irradiation time.

TABLE 2

| | fixed energy (×10⁻⁵ J/m²) | | | |
|---|---|---|---|---|
| light irradiation time | AAP | | CAP | |
| (min) | TN-PAA | P-PAA | TN-PAA | P-PAA |
| 1 | 1.629 | 1.277 | 1.725 | 1.135 |
| 3 | 3.466 | 1.788 | 3.466 | 1.564 |
| 5 | 1.755 | 1.962 | 2.934 | 1.518 |
| 8 | 2.125 | 1.651 | 2.831 | 2.015 |
| 10 | 1.281 | 1.634 | 6.926 | 2.084 |

It was confirmed that the fixed energy had different values depending on the type of the photoactive agent and the polyamic acid and the light irradiation time. In Table 2, the liquid crystal alignment layer indicated by AAP refers to an alignment layer in which TN-PAA and P-PAA are used as polyamic acid and AAP is used as a photoactive agent, respectively. In the case of AAP, the maximum value of 3.466×10⁻⁵J/m² when the light irradiation time is 3 minutes, and in the case of CAP, the maximum value of 6.926×10⁻⁵J/m² when the light irradiation time is 10 minutes. Upon light irradiation, the photoactive agent generates each radical with polyimide, AAP generates a methyl radical, and CAP generates an allyl radical, and each of these radicals attacks some bonds of the broken polyimide due to light irradiation. It is judged that CAP requires more light irradiation time to show the maximum fixed energy compared to AAP because the stability of the allyl radical is greater than that of the methyl radical. The fixed energy of the liquid crystal cells in which the photo-active agent is mixed and photo-aligned has a similar or higher value than the liquid crystal cell having only a rubbing process as a whole, and it is judged that these results show that the surface area and surface roughness of the liquid crystal alignment layer due to evaporation of carbon dioxide generated during light irradiation increases.

Table 3 shows the results of the fixed energy evaluation for the liquid crystal alignment layer using TN-PAA as the polyamic acid and performing both the rubbing process and light alignment.

TABLE 3

| | |
|---|---|
| light irradiation time | fixed energy (×10⁻⁵ J/m²) |

| (min) | AAP | CAP |
|---|---|---|
| 1 | 1.076 | 1.200 |
| 3 | 1.287 | 1.200 |
| 5 | 1.034 | 1.493 |
| 8 | 1.053 | 1.085 |
| 10 | 1.058 | 4.276 |

It was found that even though the rubbing process was added the fixed energy had a similar value to the case the rubbing process was performed to the TN-PAA. It is judged that the photoactive agent is light sensitive, so that the rubbing process does not significantly affect the increase in fixed energy. However, the light irradiation time showing the maximum fixed energy tended to be very similar to that TN-PAA and photoactive agents is used.

Table 4 shows the measurement results for the pretilt angle according to the presence or absence of a rubbing process and the type of the photo alignment agent.

TABLE 4

| | TN-PAA | | P-PAA |
|---|---|---|---|
| photoactive | Rubbing + | Only photo | Only photo |
| agent | photo alignment | alignment | alignment |
| AAP | 7~10 | 8~10 | 9~11 |
| CAP | 7~10 | 8~10 | 9~13 |

The pretilt angle tended to decrease slightly when both rubbing and light alignment were performed, but was no significant difference with that of the case of only light alignment. In addition, it can be seen that the pretilt angle is smaller than that of the case P-PAA is used when TN-PAA is used.

Table 5 shows the measurement results of the maximum dichroic ratio according to the light irradiation time. In addition, FIG. 12 shows a polar diagram.

TABLE 5

| light irradiation time (min) | AAP | CAP |
|---|---|---|
| 1 | 0.3840 | 0.1484 |
| 3 | 0.4555 | 0.2167 |
| 5 | 0.1601 | 0.6346 |
| 8 | 0.2778 | 0.2222 |
| 10 | 0.1072 | 0.1157 |

AAP showed a maximum dichroic ratio of 0.4555 at 3 minutes and CAP showed a maximum dichroic ratio of 5 minutes at 0.6346. This dichroic ratio value means that the light alignment occurs in a shorter time than the light irradiation time with the maximum fixed energy. Therefore, it can be seen that the liquid crystal alignment layer has fast alignment characteristics required for efficiency and productivity of the process.

FIG. 12 is a polar diagram of a liquid crystal cell including AAP and CAP, and shows the degree of alignment at the time of light irradiation with the maximum dichroic ratio. The liquid crystal cell was measured in a state parallel to the alignment direction and perpendicular to the direction of the polarizer. According to the results of the polarity evaluation, referring to FIG. 12, the polarizer shows the maximum absorbance at 90° and 270°, the minimum absorbance at 0° and 180°, and shows the overall peanut shape. Therefore, it can be seen that the alignment of the liquid crystal alignment layer was good.

Table 6 shows the measurement results of the contact angle and surface roughness of the liquid crystal alignment layer.

TABLE 6

| | |
|---|---|
| Type | Contact angle (degree) |

| Photo-Polyimide (P-PI) | | 20.4504 |
|---|---|---|
| Irradiated P-PI (IP-PI) | | 32.6770 |
| P-PAA + AAP | before light irradiation | 22.1825 |
| | after light irradiation | 33.0115 |
| P-PAA + CAP | before light irradiation | 23.9174 |
| | after light irradiation | 39.9966 |

P-PI increases the contact angle after light irradiation, which is due to an increase in the unsaturated C═C bond at the surface, a decrease in the oxygen group content, and an increase in the carbon group content. The surface of PI becomes more stable through light irradiation. When AAP and CAP were added to P-PAA, the contact angle was respectively increased after light irradiation. When comparing AAP and CAP, CAP makes the liquid crystal alignment layer more hydrophobic than AAP. On the other hand, when comparing TN-PAA and P-PAA, the contact angle of TN-PAA was increased to a greater extent, and through this, P-PI had larger than that of TN-PI since P-PI has more polar hydroxyl groups and hydrophobicity than TN_PI. These results are also consistent with those of the IR spectra.

The surface roughness of the liquid crystal alignment layer was increased from 0.2861 nm to 0.4645 nm after light irradiation, and referring to FIG. 13, it can be confirmed that the surface became more rough. It is judged that the surface of the liquid crystal alignment layer becomes rougher because carbon dioxide from the surface evaporates upon light irradiation. As the surface roughness of the liquid crystal alignment layer is increased, the liquid crystal alignment layer has an increased surface area and facilitates reaction with liquid crystal molecules.

As described above, AAP and CAP, which are photoactive agents according to the present invention, have an advantage in that, when added to the photo-alignment agent, there is no need for a special device in the manufacturing process of the liquid crystal alignment layer, and post-treatment is convenient. In addition, the photo-alignment agent including the photoactive agent AAP and CAP according to the present invention is sensitive to ultraviolet light of a weaker intensity than the conventional photo-alignment, thereby shortening the process time and increasing the fixed energy. It is judged to be advantageous in solving chronic problems such as image sticking. In addition, in the liquid crystal alignment layer according to the present invention, an unsaturated C═C bond is formed on the surface of the liquid crystal alignment layer after light irradiation, and the carbon content increases and the oxygen content decreases.

In addition, the liquid crystal alignment layer according to the present invention has an increased surface roughness and surface area having hydrophobicity due to evaporation of carbon dioxide an serves to make it easier for liquid crystal molecules to penetrate into an empty space.

What has been described above is only one embodiment for carrying out the photoactive agent according to the present invention, the manufacturing method of the same, and the manufacturing method of a liquid crystal aligning agent including the same, and the present invention is not limited to the above-described embodiment, and the following patents as claimed in the claims, any person having ordinary knowledge in the field to which the present invention pertains without departing from the gist of the present invention will have the technical spirit of the present invention to the extent that various changes can be made.

1. A photoactive agent having an N—O—C═O—C band and being mixed with a liquid crystal aligning agent.

2. The photoactive agent of claim 1, wherein the photoactive agent has a structural formula (a) or (b) below:

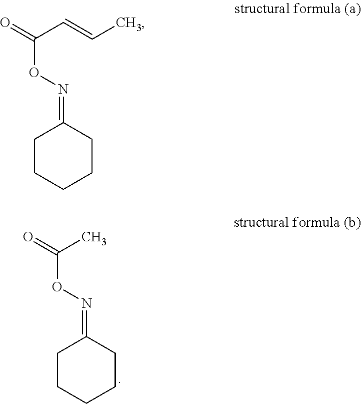

3. The photoactive agent of claim 1, wherein the photoactive agent has a structural formula (c), (d) or (f) below: 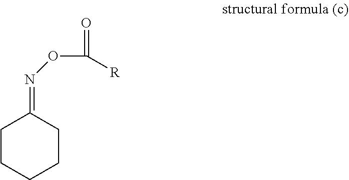
wherein R is alkyl group or cyclohexyl group of CₙH₂ₙ₊₁ having 1 to 12 carbon atoms, and n is a positive integer, 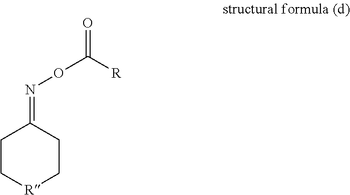
wherein R is a structure represented by the following structural formula (e) or a cyclohexyl group, R″ is alkyl group of CₙH₂ₙ₊₁ having 1 to 5 carbon atoms, and n is a positive integer, 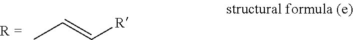
wherein R′ is alkyl group of CₙH₂ₙ₊₁ having 1 to 12 carbon atoms, n is a positive integer, and R and R″ are the same or different from each other, 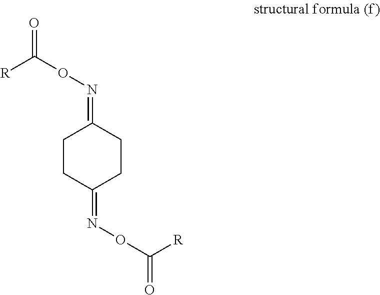
wherein R is alkyl group or a cyclohexyl group of CₙH₂ₙ₊₁ having 1 to 12 carbon atoms, and n is a positive integer.

4. The photoactive agent of claim 1,
wherein the photoactive agent of structural formula (a) is formed by the reaction of cyclohexanoneoxime with crotonic anhydride,
the photoactive agent of the structural formula (b) is formed by the reaction of cyclohexanoneoxime and acetic anhydride.

5. A manufacturing method of photoactive agent comprising:
a primary solution preparation step for preparing a primary solution by mixing and dissolving cyclohexanone oxime in a solvent;
a secondary solution manufacturing step for preparing a secondary solution by mixing anhydride in the primary solution;
a reaction step for proceeding a synthesis reaction of the photoactive agent in a reaction solution in which a catalyst is mixed with the secondary solution; and
an extraction step for extracting the photoactive agent in the reaction process.

6. The manufacturing method of photoactive agent of claim 5,
wherein the solvent comprises any one selected from the group consisting of hexane THF, DMF and DMSO.

7. The manufacturing method of photoactive agent of claim 5,
wherein the anhydride is crotonic anhydride when the solvent is hexane,
wherein the anhydride is acetic anhydride when the solvent is dimethyl formamide.

8. The manufacturing method of photoactive agent of claim 5,
wherein the catalyst is perchloric acid, sulfuric acid, para-toluenesulfonic acid, hydrochloric acid or nitric acid.

9. The manufacturing method of photoactive agent of claim 5,
wherein the extraction step proceeds to extract the photoactive agent after alternately mixing hexane and deionized water in the reaction solution in which the synthesis reaction is completed, washing several times with hexane and deionized water, separating into a hexane layer and a deionized water layer when the solvent is hexane and
wherein the extraction step proceeds to extract the photoactive agent by evaporating the ethyl acetate after mixing and diluting ethyl acetate in the reaction solution to remove the dimethyl formamide and repeatedly mixing and washing the deionized water with the reaction solution to separate the layer into an ethyl acetate layer and a deionized water layer when the solvent is dimethyl formamide.

10. The manufacturing method of photoactive agent of claim 9,
wherein the extraction step proceeds in the state that the light is blocked,
wherein the extracted photoactive agent is sealed in a container in which light and oxygen are not transmitted and stored in a vacuum state.

11. A manufacturing method of liquid crystal alignment agent comprising:
a photoactive agent mixing step for mixing the photoactive agent of claim 1 in a predetermined ratio in an alignment undiluted solution to prepare a photoactive agent mixed solution, the method comprising:
a pyrrolidinone mixing step for mixing pyrrolidinone in the photoactive agent mixed solution;
a 2-butoxyethanol mixing step for mixing 2-butoxyethanol in the photoactive agent mixed solution;
a gamma-butyrolactone mixing step for mixing gamma-butyrolactone in the photoactive agent mixed solution; and
a stirring step for stirring the photoactive agent mixed solution for a predetermined time to prepare a liquid crystal alignment agent after the gamma-butyrolactone mixing step.

12. The manufacturing method of liquid crystal alignment agent of claim 11, wherein the alignment undiluted solution is TN-PAA or P-PAA.

13. The manufacturing method of liquid crystal alignment agent of claim 11, wherein the alignment undiluted solution is a photodegradable polymer resin.
